(19) United States
(12) Patent Application Publication
(10) Pub. No.: US 2021/0282654 A1
(43) Pub. Date: Sep. 16, 2021

(54) METHODS AND SYSTEM FOR DYE-FREE VISUALIZATION OF BLOOD FLOW AND TISSUE PERFUSION IN LAPAROSCOPY

(71) Applicant: CHILDREN'S NATIONAL MEDICAL CENTER, Washington, DC (US)

(72) Inventors: Jaepyeong CHA, Washington, DC (US); Corey ZHENG, Washington, DC (US); Lung Wai LAU, Washington, DC (US)

(73) Assignee: CHILDREN'S NATIONAL MEDICAL CENTER, Washington, DC (US)

(21) Appl. No.: 17/255,731

(22) PCT Filed: Jun. 28, 2019
(86) PCT No.: PCT/US2019/039888

§ 371 Date: Dec. 23, 2020

Related U.S. Application Data
(60) Provisional application No. 62/691,386, filed on Jun. 28, 2018.

(51) Int. Cl. A61B 5/026; A61B 1/313; A61B 1/05; A61B 1/00; A61B 1/07; A61B 5/00

(52) U.S. Cl. CPC A61B 5/0261; A61B 1/313; A61B 1/05; A61B 1/00009; A61B 1/07; A61B 5/0084; A61B 1/00188; A61B 1/00174

(57) ABSTRACT

A visualization system, an apparatus, and a visualization method are provided. The visualization system includes a laparoscope, a camera operatively coupled to the laparoscope, a light source operatively coupled to an illumination port of the laparoscope, and processing circuitry. The light source is configured to output one or more light beams each at a predetermined frequency to illuminate a target area. The processing circuitry is configured to process imaging data from the laparoscope received by the camera to generate one or more images of the target area including at least one laser speckle contrast image. The laparoscope is configured to output the one or more light beams toward the target area at a distal end thereof and to collect reflected and/or scattered light from the target area via the distal end.

### CROSS-REFERENCE TO RELATED APPLICATIONS

This application claims the benefit of priority from U.S. Provisional Application No. 62/691,386 filed Jun. 28, 2018, the entire contents of which are incorporated herein by reference.

### BACKGROUND

### Field of the Disclosure

The present disclosure is related to a system and method for single port laser speckle contrast image analysis in laparoscopy.

### Description of the Related Art

Laser speckle contrast imaging (LSCI) is a noninvasive vascular/tissue perfusion imaging technique, which calculates speckle contrast from monochromatic light illumination and has been well studied in various clinical applications such as neurosurgery. In LSCI, a target is illuminated using monochromatic laser light. Speckle patterns are generated on the target due to light interference. Setups including a separate laser source from a fiber and imaging device located on the different angles may be used.

Unlike fluorescence angiography, LSCI permits a seamless visualization of blood flow and does not require injection of contrast agents. Laparoscopic implementation of LSCI faces difficulty from several technical issues including laser light source integration, fiber light guide coupling, and specular reflections from tissue surfaces. Known endoscopic LSCI systems are limited by the need for an external laser source, a short working distance or direct contact with tissue, or poor resolution and the inability to resolve individual vasculature, all of which limit their practical application to minimally invasive surgery (MIS).

For example, U.S. Patent Publication No. 2017/181636A1 entitled “Systems for imaging of blood flow in laparoscopy” by Luo et al. describes a system using a separate optical fiber to illuminate laser light. However, for minimally invasive surgery, small number of incisions would be ideal and different light sources create adverse effects on the illumination such as shadows and angle dependent uneven lights.

In techniques of endoscopic laser speckle contrast analysis, a separate laser light source to generate laser speckle patterns apart from endoscope or single port approach in contact with the tissue are used. Single port approaches in contact with the tissue have limitations on their utility in terms of fixed small field of view (no rooms for focus, magnifications and etc.), tissue damage due to the direct contact (physical contact) and no space for intraoperative procedures (only for diagnostic purpose).

The foregoing “Background” description is for the purpose of generally presenting the context of the disclosure. Work of the inventor, to the extent it is described in this background section, as well as aspects of the description which may not otherwise qualify as prior art at the time of filing, are neither expressly or impliedly admitted as prior art against the present invention.

### SUMMARY

The present disclosure relates to a visualization system. The visualization system includes a laparoscope, a camera operatively coupled to the laparoscope, a light source operatively coupled to an illumination port of the laparoscope, and processing circuitry. The light source is configured to output one or more light beams each at a predetermined frequency to illuminate a target area. The processing circuitry is configured to process imaging data from the laparoscope received by the camera to generate one or more images of the target area. The laparoscope is configured to output the one or more light beams toward the target area at a distal end thereof and to collect reflected and/or scattered light from the target area via the distal end.

In one aspect, the present disclosure also relates to an apparatus for laser speckle contrast imaging. The apparatus includes a laparoscope having an illumination port and one or more image sensors operatively coupled to the laparoscope. The laparoscope is configured to receive one or more light beams via the illumination port, output the one or more light beams toward a target area, and capture one or more images of the target area via a common path.

In one aspect, the present disclosure relates to a visualization method. The visualization method includes providing a visualization apparatus including a laparoscope, a camera operatively coupled to the laparoscope, a light source operatively coupled to an illumination port of the laparoscope; outputting one or more light beams from the light source at predetermined frequencies to illuminate a target area; capturing reflected and/or scattered light from the target area via the laparoscope; and processing the captured light to generate at least a laser speckle contrast image of the target area.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1A is a schematic that shows a visualization system according to one example;

FIG. 1B is a schematic that shows a front view of the visualization system according to one example;

FIG. 1C is a schematic that shows a side view of the visualization system according to one example;

FIG. 1D is a schematic that shows an exploded view of the visualization system according to one example;

FIG. 2A is a schematic that shows the visualization system according to another example;

FIG. 2B is a schematic that shows an image of the visualization system according to one example;

FIG. 2C is a schematic that shows a light source of the visualization system according to one example;

FIG. 2D is a schematic that shows an adjustable polarizer cap of the visualization system according to one example;

FIG. 3 is a block diagram of an imaging system according to one example;

FIG. 4 is a flowchart for a method for tissue visualization according to one example;

FIG. 5 is a schematic that shows a power output from the light source of the visualization system according to one example;

FIG. 6A is a schematic that shows a raw near infra-red (NIR) image of a white paper according to one example;

FIG. 6B is a schematic that shows a surface of normalized illumination intensity according to one example;

FIG. 6C is a schematic that shows the normalized illumination intensity for a line sampled across a center of illumination according to one example;

FIG. 6D is a schematic that shows a surface plot of normalized contrast values according to one example;

FIG. 6E is a schematic that shows a plot of the normalized contrast values along the line sampled across the center of illumination according to one example;

FIG. 7A is a schematic of a laser speckle contrast imaging (LSCI) of a flow phantom according to one example;

FIG. 7B is a schematic of a fabricated acrylic flow phantom according to one example;

FIG. 7C is a schematic that shows an in vitro phantom experimental setup according to one example;

FIG. 8 is a schematic that shows computational fluid dynamics (CFD) simulations results for a microfluidic phantom according to one example;

FIG. 9 is a schematic that shows the visualization of all channels at a 5 cm range according to one example;

FIG. 10 is a schematic that shows relative flow rates compared to the expected relative flow rates at all the channel sizes and volumetric flow rate inputs according to one example;

FIG. 11 is a schematic that shows normalized intensity values and measured flow profile according to one example;

FIG. 12 is a schematic that shows the LSCI-processed images according to one example;

FIG. 13 is a schematic that shows normalized relative flows according to one example;

FIG. 14 is a schematic that shows images of the small and mesentery according to one example;

FIG. 15 is a schematic that shows images of clamped and unclamped bowel mesentery according to one example;

FIG. 16 is a schematic that shows images of various pig organs according to one example; and

FIG. 17 is a block diagram of a computer according to one example.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically. The term “program” or “computer program” or similar terms, as used herein, is defined as a sequence of instructions designed for execution on a computer system. A “program”, or “computer program”, may include a subroutine, a program module, a script, a function, a procedure, an object method, an object implementation, in an executable application, an applet, a servlet, a source code, an object code, a shared library/dynamic load library and/or other sequence of instructions designed for execution on a computer system.

Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means “any of the following: A; B; C; A and B; A and C; B and C; A, B and C”. An exception to this definition will occur only when a combination of elements, functions, steps or acts are in some way inherently mutually exclusive.

Referring now to the drawings, wherein like reference numerals designate identical or corresponding parts throughout several views, the following description relates to a system and associated methodology for laser speckle contrast imaging (LSCI) including a single-exposure LSCI and a laparoscope. The system includes a single port common path illumination system and a camera sensor system. In other words, the system has a common path for the imaging data received by a camera of the camera sensor system and an output of the one or more lasers of the illumination system. The system may generate real-time information corresponding to a target area (i.e., illumination area). The target area may be a surgical scene including a tissue structure. No shadow areas are produced on the target area using the system.

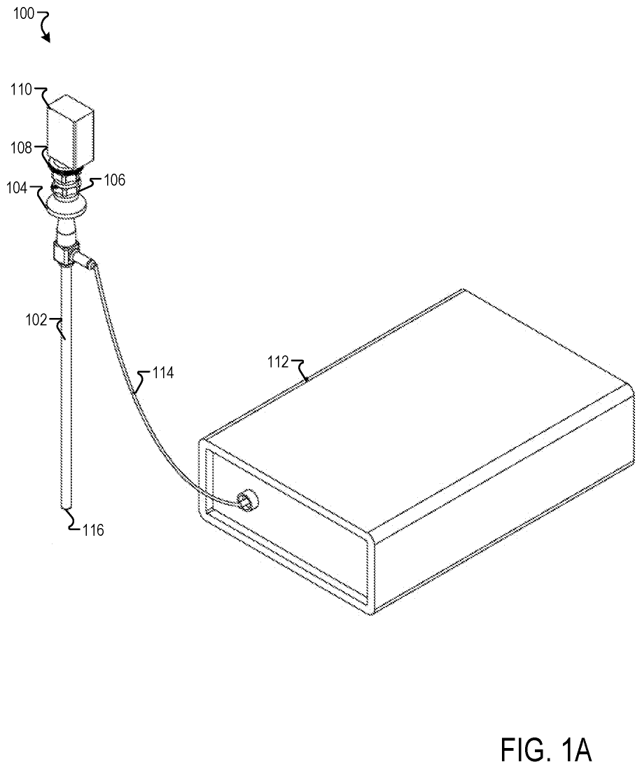

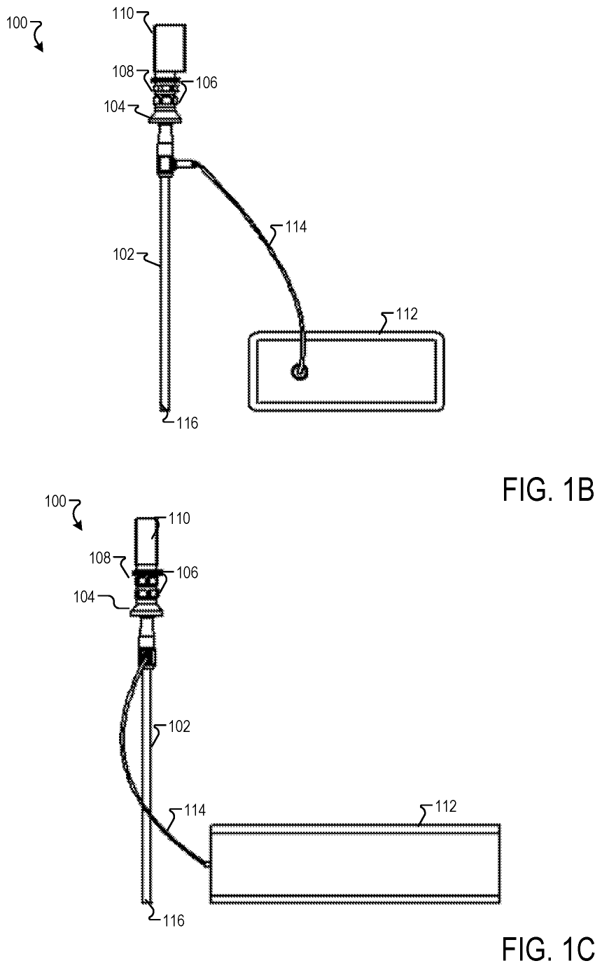

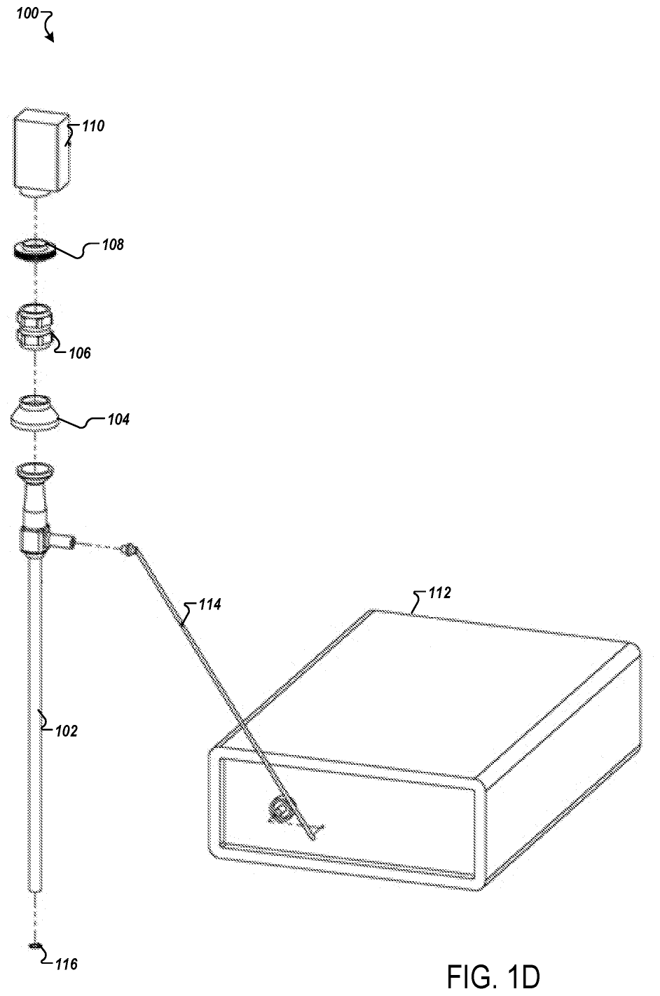

FIG. 1A is a schematic that shows the visualization system 100 according to one example. The visualization system 100 includes a laparoscope 102, a first connector 104, a lens adapter 106, a polarization state analyzer 108, a camera sensor 110, a light source 112, a fiber optic light guide 114, and a polarization state generator 116. The laparoscope 102 has an angle of rom about zero to thirty degrees. In one implementation, the laparoscope 102 may be a 0 or 30 degrees angled scope. The first connector 104 couples the laparoscope 102 and the camera sensor 110. The polarization state analyzer 108 and the lens adapter 106 are positioned between the camera sensor 110 and the laparoscope 102. The lens adapter 106 may be a focus adjustable lens adapter. The polarization state generator 108 may be attached to a first distal end of the laparoscope 102. The light source 112 generates one or more light beams having multiple wavelengths. The light source 112 generates at least one light beam having a coherent light such as a laser light beam. For example, the light source 112 includes a broadband visible light source and a near-infrared laser source. The light source 112 is connected to the laparoscope 102 via the fiber optic light guide 114. The fiber optic light guide 114 is coupled to an illumination port of the laparoscope 102. The laparoscope 102 directs the one or more light beams to the target area. The camera sensor 110 is configured to capture light reflected or scattered by the target area via the laparoscope 102. In one implementation, the laparoscope 102 includes a single illumination port. FIGS. 1B and 1C show side views of the system 100. FIG. 1D is a schematic that shows an exploded view of the system 100.

The system described herein is configured to operate up to a 5 cm working distance. In other implementations, a working distance of the system is in the range of 2 cm to 15 cm, 3 cm to 9 cm, or 4 cm to 8 cm. The working distance may be equal to 4.5 cm, 4.6 cm, 4.7 cm, 4.8 cm, 4.9 cm, 5.1 cm, 5.2 cm, 5.3 cm, 5.4 cm, or 5.5 cm. The space between the laparoscope 102 and the target area may be used for intraoperative procedures and surgical tools. The magnification of the target area may be controlled by the working distance. A closer distance from the target area magnifies the target area and allows high resolution blood vessel imaging with a spatial resolutions ranging from about 20 μm to 30 μm.

The system 100 may or may not use an externally positioned laser source.

The illumination system may be a laser illumination system that includes the light source 112. The laser illumination system may provide laser beams with different wavelengths which have a common path. The different wavelengths covers the visible spectrum and the infrared spectrum including near-infrared (NIR), short wavelength infrared (short wave infrared (SWIR)), medium wavelength infrared, and long wavelength infrared. Using multiple wavelengths permit the imaging of different layers of the tissue (or tissue structure of the illumination area). Short wavelength penetrates only a top layer of the tissue structure, while the longer wavelengths penetrate deeper into it. The shorter wavelengths are used to detect tumors. Short wavelength infrared (SWIR) laser light can penetrate deep in tissue. The more deeply penetrating longer wavelength allows the generation of information corresponding to the vasculature of lesions or other structures located more distant from the tissue surface. The system 100 uses multiple laser lights (having different wavelengths) for depth-resolved blood flow measurement. Thus, one or more of the laser light beams may be used based on the target being imaged. The laser light beams are coupled to the single illumination port of the laparoscope 102 via the fiber optic light guide 114.

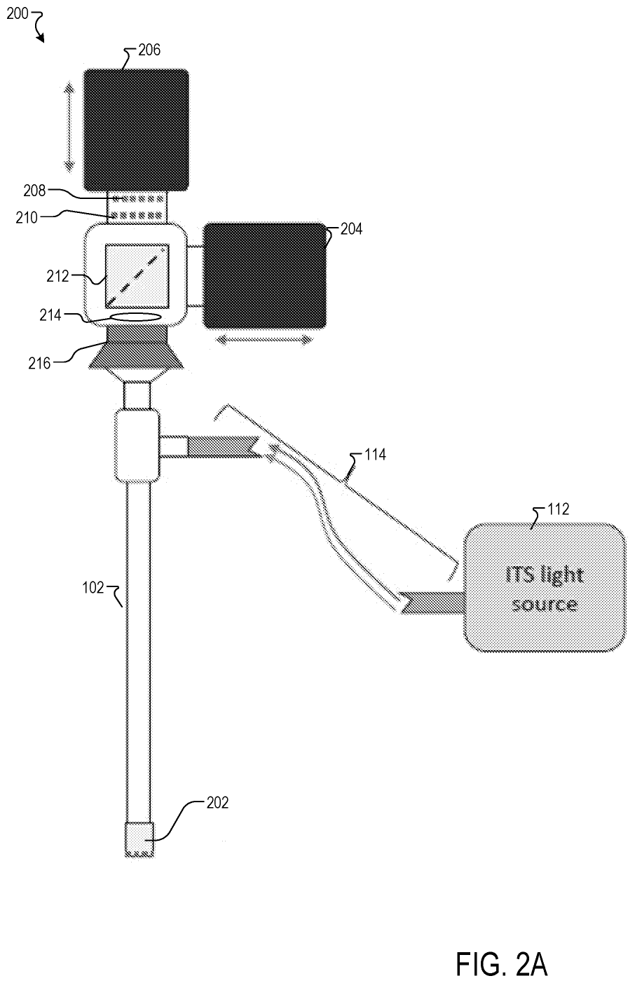

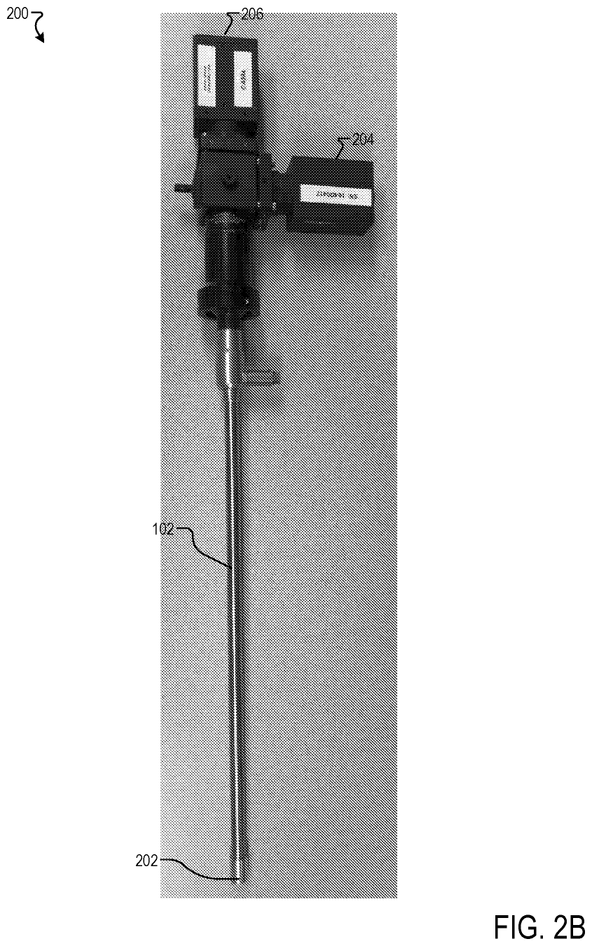

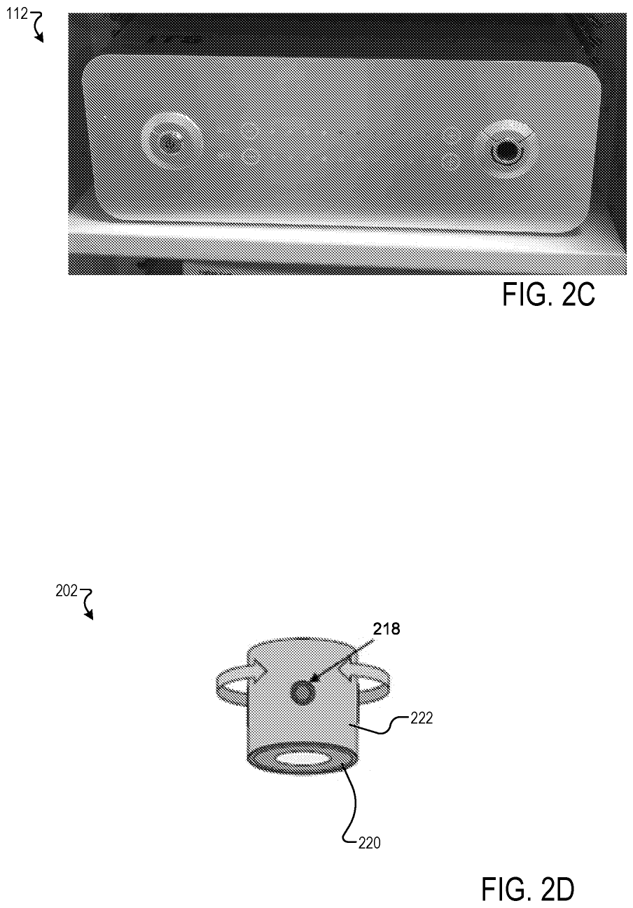

FIG. 2A is a schematic that shows a laparoscopic LSCI system 200 according to another example. The laparoscopic LSCI system 200 includes the laparoscope 102, the light source 112, an adjustable polarizer cap 202, a fiber optic light guide 114 (i.e., fiber optic cable), a first camera 204, a second camera 206, a high pass filter 208, a linear polarizer 210, a beam splitter 212, and a lens 214. The lens 214 may be one or more lenses 214. The laparoscopic LSCI system may be as described in “Dual-display laparoscopic laser speckle contrast imaging for real-time surgical assistance” by Zheng et al. incorporated herein by reference in its entirety (Biomedical optics express, Vol. 9, No. 2).

In one implementation, the laparoscope 102 is a zero-degree 10 mm laparoscope. For example, the laparoscope may be a LAPALUX Telescope from MGB (Germany). The laparoscope 102 is coupled to the first camera 204 and the second camera 206 via the beam splitter 212. The beam splitter 212 may be a 50/50 beam splitter such as model number BSW26R from THORLABS. Other beam splitter ratio may be used to control the light intensity received by each camera. The beam splitter 212 may be attached to the laparoscope 102 via a laparoscope to C-mount coupler 216. The beam splitter 212 divides the optical path to the first camera 204 and the second camera 206. The first camera 204 may be an RGB camera. In one example, the model number of the first camera 204 is HD Color Vision GS3-U3-41C6C-C FLIR made by POINTGREY. The second camera 206 may be a near-infrared (NIR) camera. The model number of the second camera 206 is GS3-U3-41C6NIR-C FLIR made by POINTGREY. The first camera 204 and the second camera 206 enable simultaneous capture of both standard color and NIR laser speckle images.

The high pass filter 208 is placed in front of the second camera 206 (i.e., NIR camera) to isolate the signal from the laser source. The high pass filter 208 may be a 800 nm long-pass filter such as FEL0800-Ø1 Longpass Filter made by THORLABS, USA. The second camera 206 operates with a 2048×2048 pixel resolution at a 16-bit color depth, and has an adjustable frames-per-second (FPS) range up to 90 depending on the selected exposure time.

The first camera 204 (i.e., RBG camera) operates with a 2048×2048 pixel resolution at a 16-bit color depth, and was set to capture at 30 FPS. The two cameras are mounted on optical plates (not shown), and can be manually focused by sliding them along their mounting rails. In one example, the optical plates may be controlled via a computer.

A crossed polarizer pair may be used to reduce specular reflection onto the second camera 206. For example, the linear polarizer 210 (such as model number LPNIRE100-B, from THORLABS, USA) is placed in front of the long-pass filter 208. A second linear polarizer 220 may be included in the adjustable polarizer cap 202.

The adjustable polarizer cap 202 is shown in FIG. 2D. The adjustable polarizer cap 202 may include the second linear polarizer 220, a cap (e.g., an aluminum cap) 222, and screws 218. The second linear polarizer 220 may be made from a wire-grid polarizing film having model number 33082 from EDMUND OPTICS, USA. The second linear polarizer 220 may be laser cut from the wire-grid polarizing film into a donut shape, covering only the laparoscope's illumination fibers and not the lens tube (e.g., using laser model Epilog Mini 40 W from TROPTEC, Austria). The adjustable polarizer cap 202 is rotated until specular reflection is minimized. The adjustable polarizer cup may be controlled via the computer.

The light source 112 is a power-adjustable dual light source including a visible spectrum (400-700 nm) high-power quad-LED and an uncollimated near infrared laser (810 nm) that shares a common optical path such as Model-L LSU, INTHESMART Incorporated, USA). The light source 112 may be FDA approved. An exemplary light source is shown in FIG. 2C. The light source 112 is coupled to the illumination port of the laparoscope 102 via the fiber-optic light guide 114.

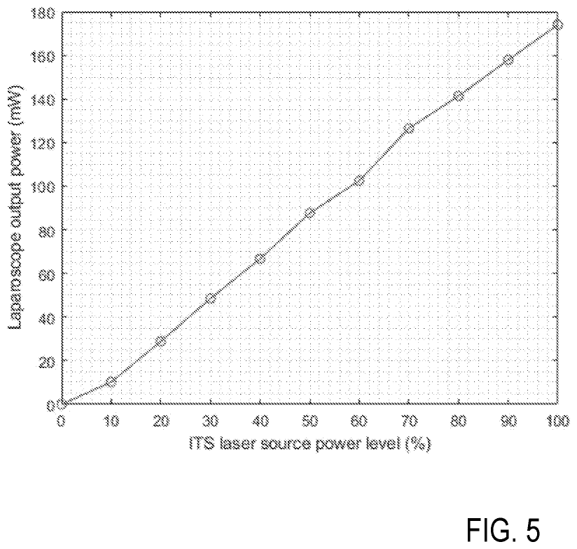

The light power can be adjusted from 0-100% power in predetermined steps (e.g., 1%, 5%, 10%, 15%, or 20%). In one example, laser power measured at 1 cm from the tip of the laparoscope 102 is set to be no greater than 40 mW, corresponding to a maximum power setting of 30% of the power of the light source 112. In one example, the exposure times of the cameras are set to a range between 8-25 ms to adjust for a received signal. The spot power output from the laparoscope tip for each laser source setting is shown in FIG. 5.

The Field-of-View (FoV) and resolution of the system are measured using a resolution target such as model R3L3S1P from THORLABS (USA) and a 1 cm square grid paper. The FoVs for a 5, 2, and 1 cm distance were approximately 7 cm, 3.5 cm, and 2 cm respectively, and the resolutions were 125 μm, 70 μm, and 28 μm, respectively.

The visualization system may further include one or more optical elements to even the illumination region.

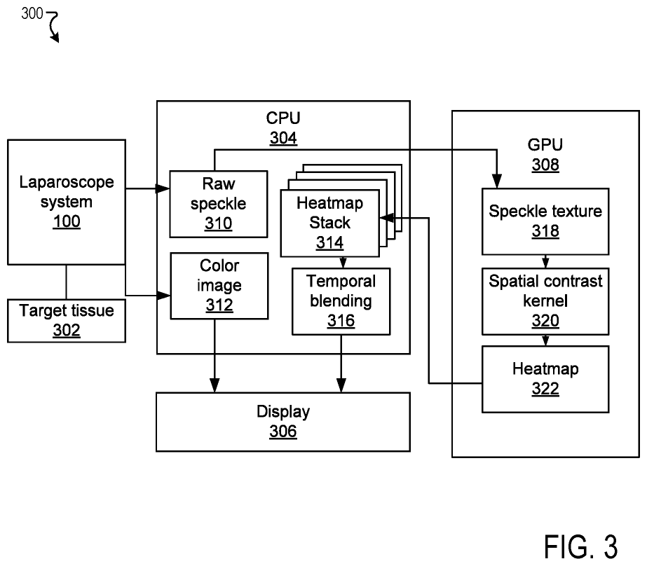

FIG. 3 is a block diagram of an imaging system 300. The imaging system 300 includes the visualization system 100, a central processing unit 304 (CPU), and a graphics processing unit 308 (GPU). The CPU 304 and the GPU 308 may be integrated into one or more computers. The visualization system 100 collects data from a target tissue 302. The visualization system 100 includes one or more near infrared cameras, one or more color cameras, and one or more polarimetric cameras. The CPU 304 receives and processes data from the visualization system 100. For example, the CPU 304 receives raw speckle data 310 from the NIR camera. The CPU 304 receives data from the one or more color cameras and generates a color image module 312. A fluorescence image may be generated from the one or more near infrared cameras. Polarimetric data may be received from the one or more polarimetric cameras. The GPU 308 may include a speckle texture module 318, a spatial contrast kernel module 320, and a heatmap module 322. The raw speckle data 310 are processed in the speckle texture module 318. The heatmap module 322 outputs to a heatmap stack which is processed in the temporal blending module 316 as described later herein. Outputs from the color image module 312 and the temporal blending module 316 are output via display 306. The spatial contrast kernel may have a 5×5 or a 7×7 sliding window.

The display 306 may be located in the same room as a target area being imaged (e.g., surgical site) or in a remote location from the target area allowing for remote surgical procedures.

The modules described herein may be implemented as either software and/or hardware modules and may be stored in any type of computer-readable medium or other computer storage device. For example, each of the modules described herein may be implemented in circuitry that is programmable (e.g. microprocessor-based circuits) or dedicated circuits such as application specific integrated circuits (ASICS) or field programmable gate arrays (FPGAS). In one embodiment, a central processing unit (CPU) could execute software to perform the functions attributable to each of the modules described herein. The CPU may execute software instructions written in a programing language such as Java, C, or assembly. One or more software instructions in the modules may be embedded in firmware, such as an erasable programmable read-only memory (EPROM).

In some implementations, the processes associated with each of the modules may be performed by one or more processors of a server or other computing resources, which can include cloud computing resources.

The processor may be included in the one or more cameras (e.g., camera 110).

Imaged speckles are converted, in the GPU 308, to a map of speckle contrast by applying equation 1 over some rolling pixel window:

$$K = \frac{σ}{〈 I 〉} \qquad ( 1 )$$

where K is the speckle contrast, σ is the standard deviation of the intensity over the window, and <I> is the mean intensity over the window. The pixel window can be purely spatial, (a square region of pixels from a single image), temporal (the same pixel over multiple frames in time), or a combination of the two, spatiotemporal (a square region of pixels over multiple frames) as would be understood by one of ordinary skill in the art.

The laser speckle contrast is not directly proportional to the blood flow velocity v, and is instead typically converted to the correlation time τ_{c}, which is assumed to be inversely proportional to flow:

$$v ∞ \frac{1}{{τ}_{c}} \qquad ( 2 )$$

The speckle contrast is related to τ_{c} through equation (3):

$${K}^{2} = β ( \frac{{τ}_{c}}{T} + \frac{{τ}_{c}^{2}}{2 {T}^{2}} [ exp ( \frac{2 T}{{τ}_{c}} ) - 1 ] ) \qquad ( 3 )$$

where K is the speckle contrast, τ_{c} is the correlation time, T is the shutter speed, and β is a correction factor that accounts for polarization and differences between the detector and speckle size. In theory, τ_{c} can be quantitatively related to the absolute blood flow rate. However, many simplifying assumptions are made in the formation of equations 2 and 3. Therefore, in a real-world application, determination of absolute blood flow is difficult, restricting the application of LSCI to relative comparisons. Thus, in one implementation, the constant β can be ignored.

In one implementation, the imaging system 300 described herein utilizes a 7×7 spatial window for processing. Because of movement, scattering from tissue, reflection, and depth of blood vessels, accurate relative comparison is impractical in vivo, and τ_{c} is not determined. A 3×3×10 is utilized for a phantom study spatiotemporal window for increased spatial resolution as described later herein. τ_{c} is determined in order to better characterize the ability of the system described herein to determine flow.

In cases where integration time T is much longer than correlation time τ_{c} (τ_{c}/T>100), the inverse the of squared contrast can be approximated to be proportional to the flow velocity.

$$\frac{1}{{K}^{2}} α v \qquad ( 4 )$$

In vivo values for τ_{c} typically lie between 100-400, within an acceptable range for the use of this approximation.

The system described herein implements equation (4) to relate the speckle contrast to some measure of velocity. Two different kernels to produce speckle contrast values are used. In real-time in vivo measurements, a 7×7 spatial window implemented. The 7×7 spatial window has been widely settled upon as a good medium between spatial resolution and accuracy of estimated speckle contrast. Additionally, the real-time system described herein is configured to temporally blend a user-defined number of flow maps together to increase signal to noise ratio.

Blood velocity, vessel diameter, vascular blood flow, depth of the vessel in the tissue, length of the vessel, vessel tortuosity, type of the blood vessel may be determined from speckle contrast as would be understood by one of ordinary skill in the arts. For example, the depth of the blood vessel may be obtained using LSCI at multiple different wavelengths. The vessel is resolved based on the LSCI image obtained at each wavelength relative to other LSCI images obtained at other wavelengths. For example, a first LSCI image is obtained using a first light laser beam having a first wavelength in the NIR. A second LSCI image is obtained using a second light laser beam having a second wavelength in the SWIR. The depth of the vessel in the tissue is then determined based on the first LSCI image and the second LSCI image. As described previously herein, both the first and the second laser beam propagate through the laparoscope via the single illumination port.

For the in vitro measurements described herein, a 3×3×10 spatiotemporal window for increased spatial resolution is used. Different methods of computation for the in vivo and in vitro are implemented due to presence of organ secondary to respiration and cardiac pulsation. This also accounts for the motion encountered during surgery with the movement of the laparoscope 102. Using a fixed spatiotemporal kernel may result in motion artifacts if the movement between frames is large. Blending spatial windows as described herein allows the user to adjust for an appropriate number of frames (large number of frames for increased signal, no blending for lower latency). The spatial window size is set to 7×7 to ensure adequate sampling even if the user chooses to not blend any frames. In in vitro, a spatiotemporal kernel for increased spatial resolution is selected due to the lack of movement as described previously herein.

Most LSCI systems display post-processed images due to the computational time required for serial, high-resolution image processing on the CPU 304. However, for clinical relevance, LSCI processing and visualization must be performed in real-time. The independence of each speckle contrast calculation enables parallelization of the problem onto the GPU 308, massively accelerating processing speeds.

Each high-resolution (2048×2048 pixels) image acquired from the NIR camera is transferred as a normalized 32-bit floating point array to the texture memory of the GPU 308. Speckle contrast across the image is calculated using a sliding spatial window (e.g., of 7×7 or 5×5 pixels) in the spatial contrast kernel module 320. The resulting speckle contrast array is normalized and heat-mapped as a 32-bit packed integer representing RBGA channels in the heatmap module 322. Heat maps are then copied from the device back to the host machine and stored in a dynamic buffer capable of holding up to 30 of the most-recently computed heat maps (e.g., heatmap stack 314). Stored heat maps are used by the temporal blending module 316 for equally-weighted temporal blending of the alpha channels to increase SNR.

The final image may be displayed to the user through a user interface (e.g., a graphical user interface (GUI) such as an OpenGL front end GUI, STARCONTROL). The user has control over adjustable parameters including the colormap alpha, colormap gamma, camera exposure time, camera FPS, and the like. The adjustable parameters may be controlled via the GUI. In one implementation, the computer may be equipped with an Intel Core i7-4770K Processor, 16 GB RAM, and a Nvidia GeForce GTX 1060Ti 6 GB graphics card. At this specification, the system is capable of operating at 89 FPS, limited by the camera, with an 11.13 ms processing time per frame. The GPU implementation performed approximately 67.2 times faster than a CPU-only approach, which had a processing time of 748 ms per image.

In one implementation, the adjustable parameters may be learned using artificial intelligence techniques. The adjustable parameters may also include a field of view and a focus of the camera.

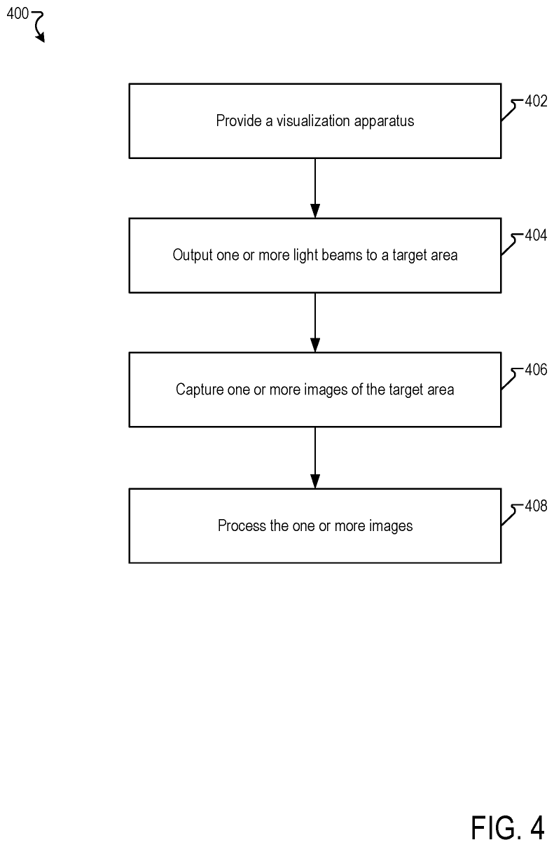

FIG. 4 is a schematic that shows a processing flowchart 400 for the system described herein according to one example.

At step 402, a visualization apparatus is provided. The visualization apparatus includes a laparoscope, a camera operatively coupled to the laparoscope, and a light source operatively coupled to an illumination port of the laparoscope as described previously herein. The light source is configured to output one or more light beams at preset frequencies to illuminate a target area.

At step 404, the laparoscope outputs the one or more light beams toward the target area. The one or more light beams may include one or more laser beams having different frequencies (wavelengths) via the single illumination port of the laparoscope.

At step 406, image data are captured via the camera coupled to the laparoscope.

In one implementation, one or more of a field of view, a magnification, and a spatial resolution of a view are adjusted when the image data are captured. The working distance may be varied to determine speckle pattern variations in a longitudinal axis of the laparoscope 102. Further, the processing circuitry may auto-focus during operation based on the determined speckle pattern variations.

In one implementation, the method includes adjusting a cross-polarizer to remove reflection artefacts.

At step 408, the image data from the laparoscope are processed as described previously herein. For example, computational compensation techniques may be implemented to even the illumination region. Further, image registration techniques may be implemented to compensate for large scale movement and hand held tool motion.

In one example, the image data are processed in real time to generate precise and accurate tissue information of mobile deformable tissue. Thus, the precise and accurate tissue information may be used in real time decision support. The tissue information is displayed in real time via a display of the system 100. A user may adjust (or select) the wavelength of the laser beam based on the displayed tissue information. For example, the user may activate a laser beam having a wavelength in the SWIR spectrum to generate information associated with deep tissue. Depth-resolved blood flow measurement may also be determined and displayed in real time. The laser beams may be controlled via the user interface.

In addition, a surgical scene for vasculature, tissue perfusion, and other critical particulate structures (e.g., lymph nodes, tumor tissues, and the like) is automatically generated by the processing circuitry in real-time. The surgical scene is displayed in real time via the display 306. Further, the surgical scene may be stored (recorded) for playback later.

In one example, when a polarized laser light beam is used (i.e., light source has a polarized pattern) or one or more polarizers are included in the system, a blood flow/tissue perfusion map of the surgical scene may be generated and displayed. The user may control the wavelength of the one or more light beams to compare relative perfusion of different tissue zones.

Although the flow chart shows specific orders of executing functional logic blocks, the order of executing the block blocks may be changed relative to the order shown, as will be understood by one of ordinary skill in the art. Also, two or more blocks shown in succession may be executed concurrently or with partial concurrence.

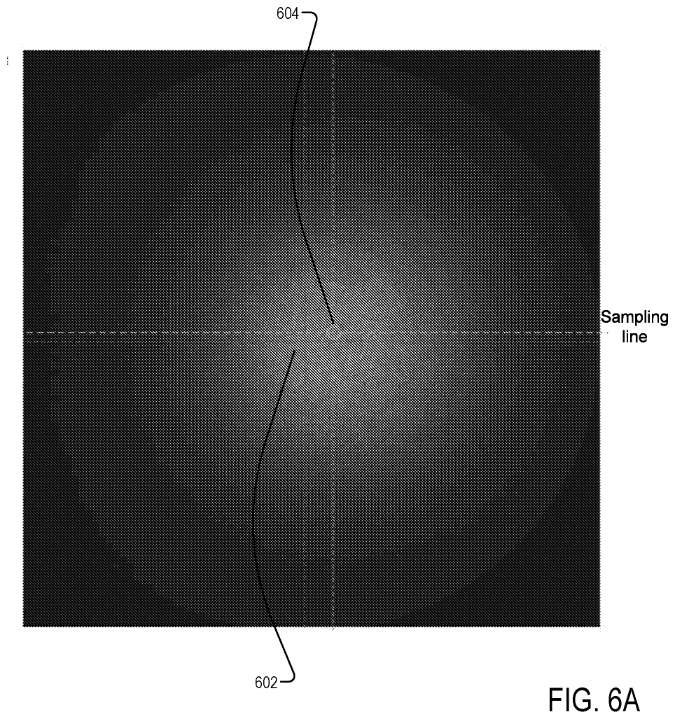

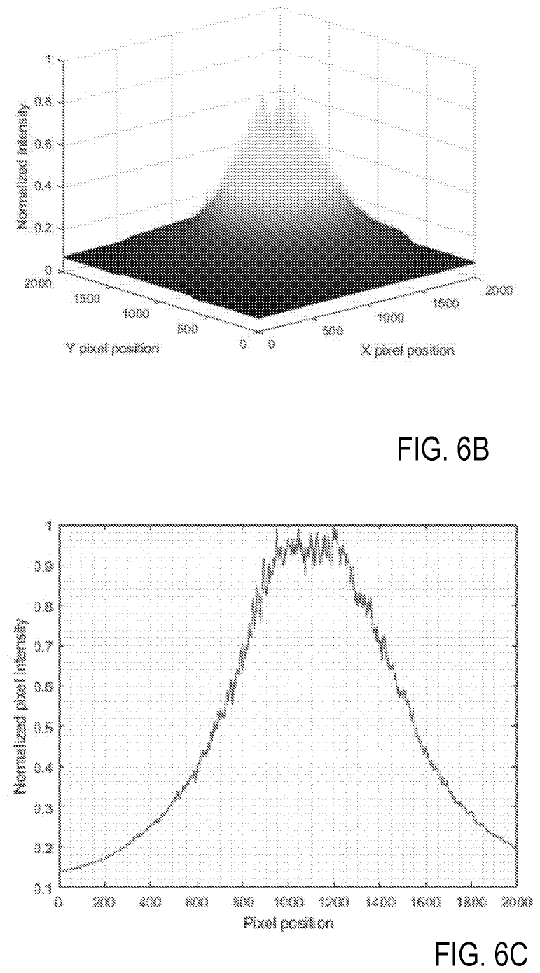

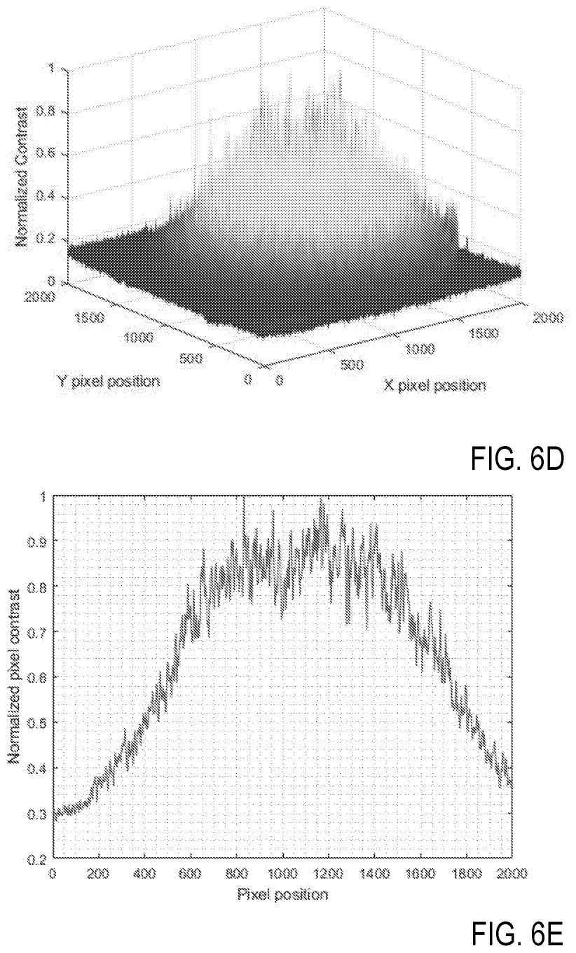

FIGS. 6A-6E show the illumination distribution of the laparoscope system at a 5 cm distance according to one example. The laparoscope 102 was oriented normal to a flat paper surface at a 5 cm distance. As shown in FIG. 6A, a center of illumination 604 is offset from an actual laparoscope axis 602. Additionally, the illumination is not even, and is heavily biased to a central region. A surface plot of the intensity versus pixel coordinate is shown in FIG. 6B, and the profile of intensities sampled across a horizontal line running through the center of intensity (labeled as “Sampling line” in FIG. 6A) is shown in FIG. 6C. The intensity drops by over 50% about 500 pixels from the center of illumination and indicates that the lighting is very uneven.

To determine the effects this concentrated illumination might have on speckle contrast, the speckle contrast was calculated according to equation (1) using a 7×7 spatial window. A surface plot of the resulting contrast is shown in FIG. 6D, and a profile of the contrasts sampled across “Sampling line” is shown in FIG. 6E. A uniform distribution of speckle contrasts with respect to pixel position is expected given that the surface is flat, non-moving, and is of a consistent material. However, speckle contrast is biased by the mean light intensity, which can affect results.

To illustrate the capabilities of the system described herein, exemplary results are presented.

In order to characterize the ability of the system described herein to resolve fine vessels and image relative flow rates with values similar to those in vivo, a microfluidic phantom was designed and fabricated.

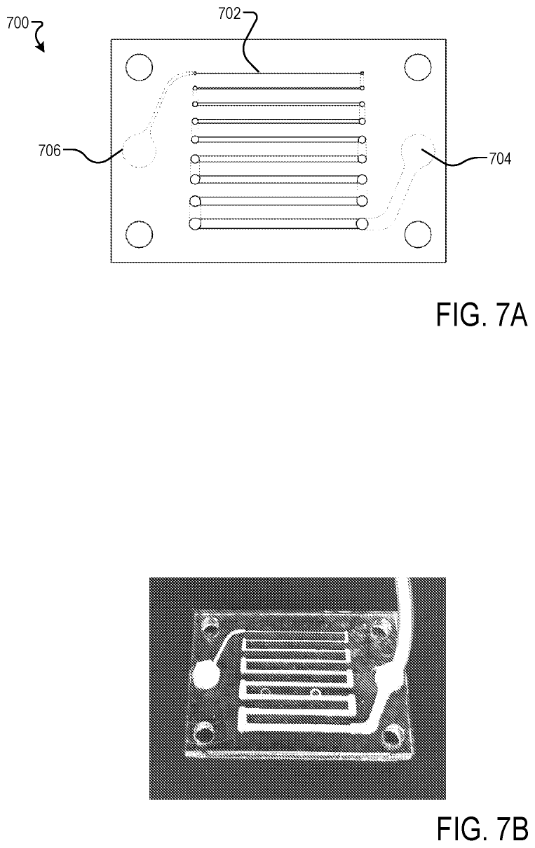

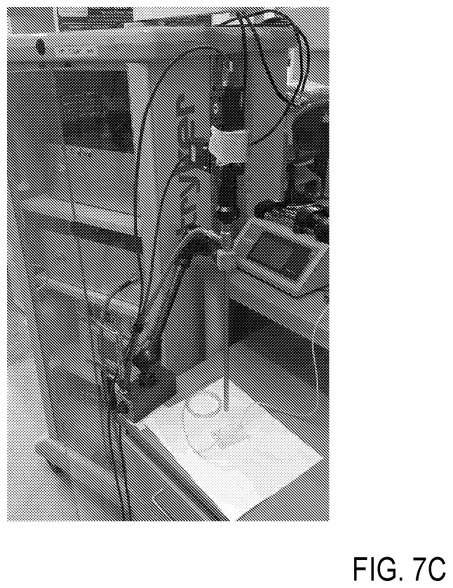

FIG. 7A is a schematic of the microfluidic phantom 700 according to one example. The microfluidic phantom includes rectangular channels 702 ranging between 0.2-1.8 mm in width. The microfluidic phantom 700 also includes an entry port 704 and an exit port 706. In one example, the widths of the channel vary in 0.2 mm steps. Channel widths are selected to approximate the diameter range of vessels commonly encountered during surgery.

The phantom is comprised of cast acrylic sheets laser-machined using a commercial laser engraver (such as TROTAC, Epilog Mini, 40 w). Channels are created using through cuts on 1/16-in. thick layers, and the width of the channels ranged from 0.2-1.8 mm in increments of 0.2 mm as described previously herein. In order to seal the phantom, the channels are sandwiched between a 1/16-in. thick acrylic cover and a 3/16-in. thick acrylic base. The stack of acrylic layers is then clamped between two aluminum plates, and the entire assembly is placed on a hotplate and heated to 150° C. for one hour, allowing the acrylic to bond and seal the phantom. Afterwards, the assembly was allowed to cool to room temperature, and then unclamped and the phantom removed. Finally, holes for tubing were drilled, and the phantom was coupled to a syringe pump (such as Pump 11 Elite Infusion/Withdrawal Programmable Single Syringe, HARVARD APPARATUS, USA) via poly-vinyl Chloride (PVC) tubing. The fabricated phantom is shown in FIG. 7B.

The microfluidic phantom 700 was infused with a 4.5% v/v dilution of Intralipid 30% and water, which roughly approximates the scattering properties of whole blood at 810 nm.

Computational fluid dynamics (CFD) may be performed on the phantom using computational tools such as SOLIDWORKS Flow Simulation® to determine the corresponding flow velocities in each channel. The fluid is approximated as water, and the channel roughness is estimated at 0.5 μm.

For each channel, the flow speed was sampled from a single point, centered about the width and length of each channel at a depth of ¼ the channel thickness, approximately 0.4 mm below the top surface. The resulting flow speeds of the center point ranged from 0-52.55 mm/s, depending on the cross-sectional area of the channel, which reflects a practical range of in vivo blood velocities. The syringe pump was then set to infuse at the same volumetric rates for the in vitro experiment (i.e., 0, 0.2, 0.4, 0.6, 0.8, and 1.0 mL/min). Corresponding average flow velocities range from 1.17-52.49 mm/s depending on the cross-sectional area of the channel, which reflects a practical range of in vivo blood velocities.

For imaging, the laparoscope tip is fixed normal to the phantom surface at a 5 cm distance using a table-mounted positioning arm (such as Articulated Holder FAT MA61003, NOGA, Israel). This represents atypical working distance a 10 mm laparoscope would be held from tissue during minimally invasive surgery (MIS). In one implementation, the NIR camera exposure time is set to 8 ms, and images are processed using a 3×3×10 spatiotemporal kernel. The experimental setup is shown in FIG. 7C.

All procedures were performed in an animal research facility under the approval of the Institutional Animal Care and Use Committee at Children's National Health System (protocol #30597).

Four female 250-300 g Sprague-Dawley rats from Charles River Laboratories (Wilmington, Mass., USA) were used. Anesthesia was induced using 3% isoflurane and maintained using intramuscular injections of 2 mg/kg Xylaxine and 75 mg/kg Ketamine. Rats were placed in a supine position and a mid-line laparotomy was performed to expose abdominal organs. Because of the small size of the rat's organs, the laparoscope was supported above the rat at distances of 1.5-3 cm from the tip to the regions of interest (ROIs) to better capture the organs within the imaging field-of-view and increase image resolution.

Bowel ischemia was created by clamping a segment of the small intestine with two clamps, placed across the bowel, occluding the arterial arcade, while a third clamp was placed on the feeding mesenteric vessel. Only the mesenteric clamp was secured and removed during the experiment to occlude and reperfuse the bowel section. Occlusion duration was limited to a 30 second window, with at least 60 seconds between additional clamping for recovery.

Real-time LSCI results were recorded during the experiment. Imaging was performed both with and without the polarizers (i.e., the linear polarizer 210 and the adjustable polarizer cap 202) at an exposure time between 10-25 ms. All animals were appropriately euthanized after experimental completion.

Pig Study Protocol (n=2)

All procedures were performed in an animal research facility under the approval of the Institutional Animal Care and Use Committee at Children's National Health System (protocol #30591).

To better correlate to a MIS experience, laparoscopic surgeries in swine were performed. Two 25-30 kg female Yorkshire pigs from Archer Farms (Darling, Md., USA) were used for the experiment. Pigs were sedated using an intramuscular injection of Xylazine and Ketamine, and anesthesia maintained using 2.5% isoflurane. A 12 mm trocar was placed at the umbilicus as the camera port. The abdomen was insufflated at 8 mmHg with CO₂. Laparoscopic LSCI was used to image perfusion of various structures at 10-25 ms exposure times, including the bowel, abdominal wall, and gallbladder. Owing to the difficulty in inserting the laparoscope through the trocar with the polarizers attached and fog forming on the polarizers in the abdomen, the polarizers were not used during the pig study.

Results

The system described herein is able to differentiate fluid flow rates and vessel sizes mimicking those found in vivo at a 5 cm working distance and exposure time of 8 ms. The microfluidic phantom 700 contains channels ranging from 0.2-1.8 mm in width, reflecting typical encountered blood vessel diameters as described previously herein. The microfluidic phantom 700 was driven at 6 volumetric flow rates from 0-1.0 mL/min, which corresponds to flow velocities from 1.17-52.49 mm/s, which covers the expected ranges for in vivo blood flow.

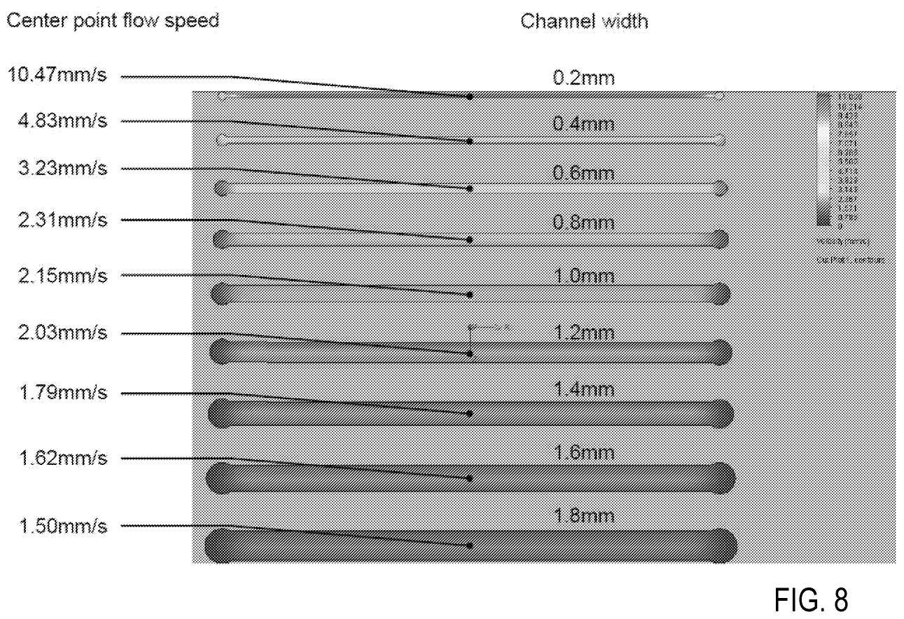

A CFD simulation was performed to determine the expected flow speed in each channel. The flow speeds were calculated by sampling a single point in each channel. The points were centered about the width and length of each channel at a depth of ¼ the channel height, approximately 0.4 mm. The results for a 0.2 mL/min volumetric input are shown in FIG. 8. The resulting flow speeds across all the channels and volumetric flow rates cover a range of 0-52.55 mm/s, which approximates the expected range for the in vivo blood flow. A detailed list of the flow speeds under each condition is listed in Table 1.

TABLE 1
Calculated flow speed (mm/s) in each channel for each of the 6
volumetric flow rates
Expected flow velocity (mm/s) in each phantom channel

| | | Channel width (mm) | | | | | | | | |
|---|---|---|---|---|---|---|---|---|---|---|
| | | 0.2 | 0.4 | 0.6 | 0.8 | 1.0 | 1.2 | 1.4 | 1.6 | 1.8 |
| Volumetric rate | 0 | 0 | 0 | 0 | 0 | 0 | 0 | 0 | 0 | 0 |
| (mL/min) | 0.2 | 10.47 | 4.83 | 3.23 | 2.31 | 2.15 | 2.03 | 1.79 | 1.62 | 1.50 |
| | 0.4 | 21.02 | 9.67 | 6.45 | 4.62 | 4.30 | 4.07 | 3.58 | 3.24 | 3.01 |
| | 0.6 | 31.53 | 14.50 | 9.68 | 6.93 | 6.44 | 6.10 | 5.36 | 4.85 | 4.51 |
| | 0.8 | 42.04 | 19.36 | 12.09 | 9.25 | 8.59 | 8.14 | 7.16 | 6.47 | 6.01 |
| | 1.0 | 52.55 | 24.22 | 16.13 | 11.57 | 10.73 | 10.17 | 8.94 | 8.09 | 7.51 |

The laparoscope 102 was fixed normal to the surface with at a distance of 5 cm. Each captured image was processed using a spatiotemporal window of 3×3×10 to calculate speckle contrast. Subsequently, the flow velocity in arbitrary units (referred to herein as laser speckle perfusion units) was calculated using the inverse of squared speckle contrast described in equation 4.

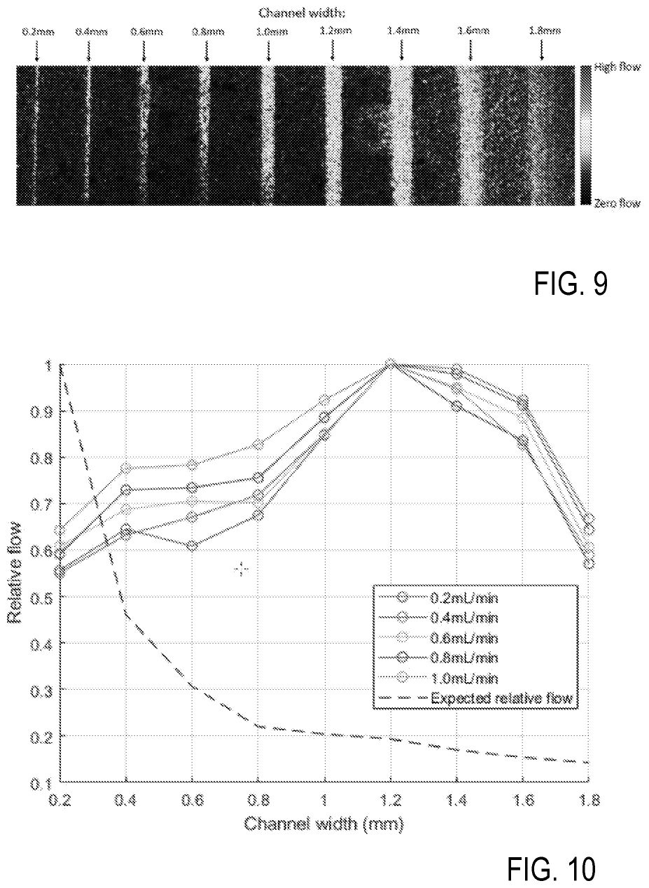

For the smallest nonzero flow rate of 0.2 mL/min, all channels are visualized. The 1.8 mm channel is at the edge of the illumination range of the laparoscope and exhibits a notable decrease in clarity. The visualization of all channels at a 5 cm range at 0.2 ml/min is shown in FIG. 9.

In one implementation, a lookup table to determine τ_{c} using the relationship described in equations 2 and 3 may be used. The lookup table may be stored in a memory of the computer and/or a cloud based database.

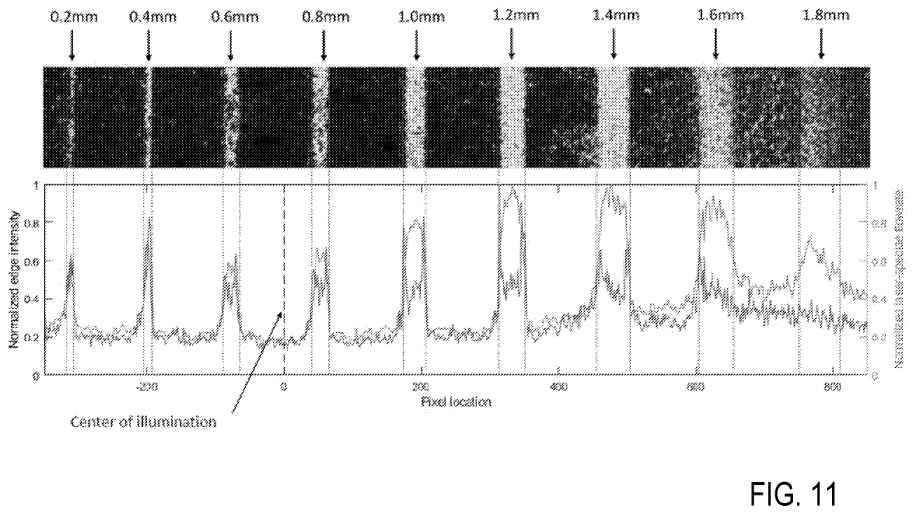

Flow in laser speckle perfusion units was then calculated from a rectangular region centered within the channel. The measured flow was normalized and compared to the expected relative flow based on the CFD computed flow speeds. This was repeated for each of the 5 nonzero volumetric flow inputs, and is shown in FIG. 10. The measured flow profiles are shown in FIG. 11.

FIGS. 10 and 11 demonstrate that the relative flow rates of the channels do not follow the expected trend calculated by the CFD. This is a restriction of the system described herein contrast values are heavily biased by the distribution of light from the laparoscope. As described previously herein, the contrast across a stationary, uniform white piece of paper is biased by the light distribution from the laparoscope 102 (FIGS. 6A-6E).

This point is best illustrated by the 1.6 and 1.8 mm channels, which are more difficult to differentiate from background noise. This is quantified by passing a 5×5 gaussian derivative kernel in the x-direction across the array of laser speckle perfusion values used to generate FIG. 9. This result in the detection of vertical edges, and the resulting edge signals are normalized and shown in FIG. 11 for the 0.2 mL/min volumetric input data. As shown in FIG. 11, the 1.8 mm channel has no clear edges, and the 1.6 mm channel does not have a clear right-side edge. Relating FIG. 6C to FIG. 9, the 1.6 and 1.8 mm channels are located further than 500 pixels from the illumination center, where the lighting intensity has dropped by over 50%. Furthermore, the fluid in the smaller channels should have a higher velocity than the fluid in the larger channels and therefore a brighter signal, but the dependence of the speckle calculations on the illumination from the laparoscope causes visual misrepresentation of flow. The 1.2 mm channel appears to have the fastest flow despite being relatively large. The system is limited by the small numerical aperture of the laparoscope light guide and the relatively short distance from laser to medium, both of which cause the light to be highly concentrated in the center of a small illumination area.

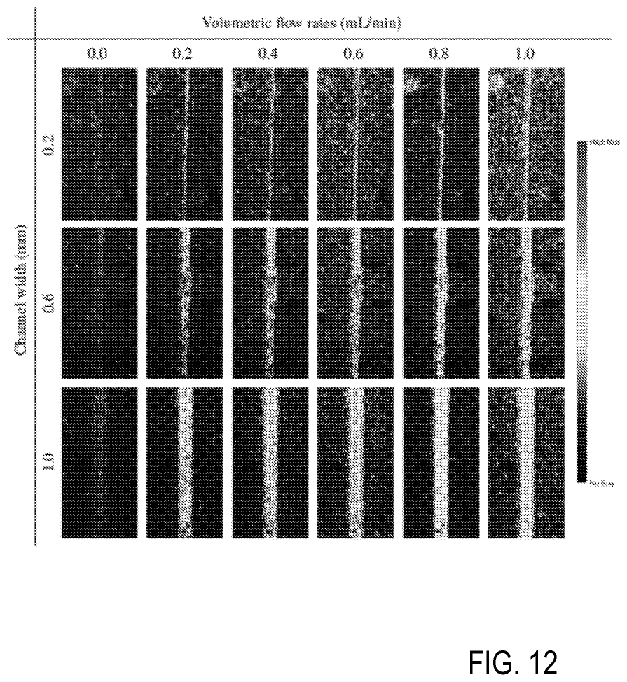

When the same locations are compared over multiple frames in time, the appropriate relative flow rates are shown. Individual examination of each channel separately over the range of the six flow rates provides a qualitative and visual indication of the increasing flow for all the channels except the 1.6 and 1.8 mm channels, which are limited by the illumination intensity. The processed images for the 0.2, 0.6, and 1.0 mL channels are displayed in FIG. 12. The granules present in some of the images are probably owing to the vibration from the laparoscope.

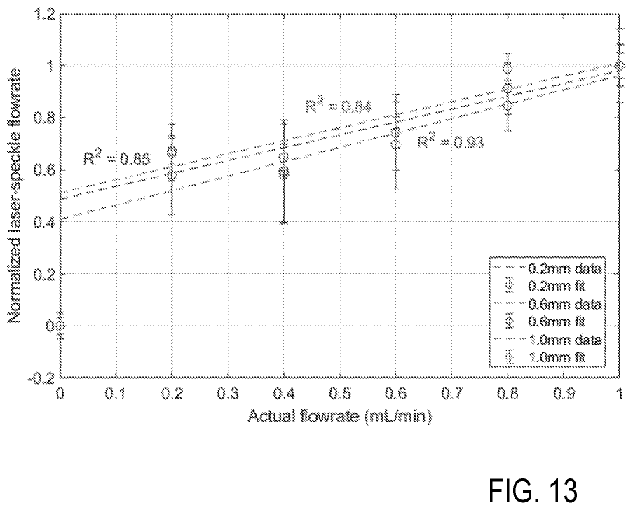

A square region of pixels centered in the channel is selected. The mean of the laser speckle perfusion in the selected region is calculated. At 0 mL/min, measurements of the laser speckle perfusion units (1/K²) are larger than that of the solid background, so that the channels are still visible when there is no flow (as shown in FIG. 12). This is because in comparison to the solid background, the fluid in the channels still has a slight movement from Brownian motion. To appropriately compare the relative flow rates for each channel, the calculated velocities are shifted by the zero offset and then normalized. There is a jump between the 0 and 0.2 mL/min input flow rates, but for non-zero flow rates, the calculated relative flow rates in each channel appear to linearly increase with the actual flow rate (as shown in FIG. 13). Deviations from the ideal relationship can be explained from the nature of single-exposure LSCI, which remains linear for smaller relative changes in flow, but breaks down in linearity for larger changes in flow rate due to interference from static scatters.

Rat Bowel Ischemia

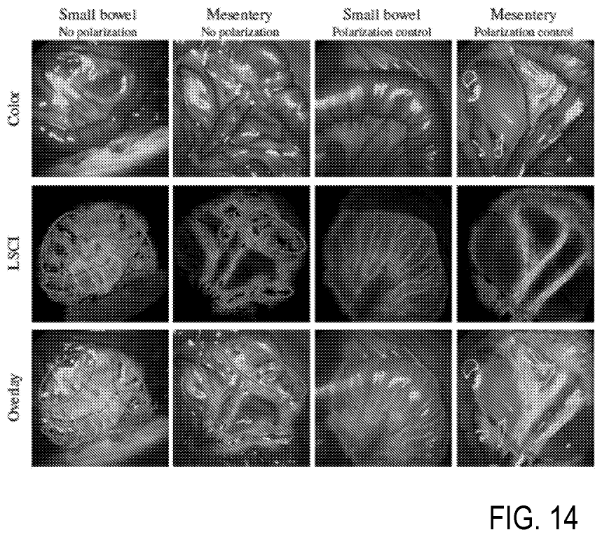

Imaging of various rat organs demonstrates that the system described herein is able to acquire color images, LSCI processed images, and overlaid images for an augmented surgical field-of-view in real time. LSCI images captured using the system described herein clearly highlight vascular structures. The effectiveness of polarization control is also demonstrated. FIG. 14 shows LSCI-processed images of the small bowel and mesentery without polarization control. Compared to images of the same regions with polarization control, the unpolarized LSCI images have many shadow spots, circled in FIG. 14, which are a result of saturated pixels that are omitted from the final visualization.

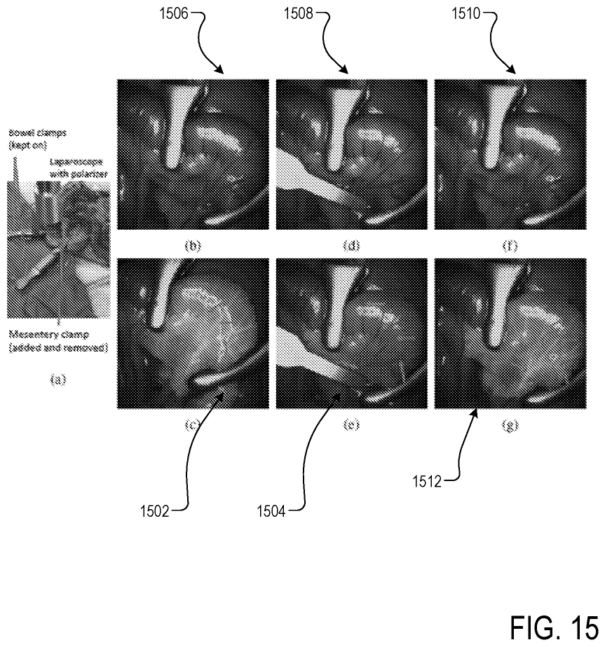

The system described herein distinguishes between normal and ischemic tissues before early changes in tissue color fully developed. In this experiment, clamps to halt flow in a section of small bowel are used (as shown in FIG. 15). Images were taken before clamping the mesentery vessel, 5 seconds after clamping, and 5 seconds after releasing the clamp. When observing purely color images, there are no visual differences between clamped and unclamped tissues. However, LSCI-processed images reveal obvious differences in flow. Vessels highlighted in the laser-speckle overlaid images 1502 shown in FIG. 15 are no longer highlighted during occlusion in image 1504, and a broad decrease in blue hue outside of vessels indicates a widespread decrease in perfusion across the occluded tissue. When comparing the corresponding color images before, after, and during clamping, there are no visual indications whether or not occlusion is occurring (image 1506, image 1508, and image 1510). After the clamp is released, image 1512 shows that the vessels reappear.

Pig Minimally Invasive Surgery

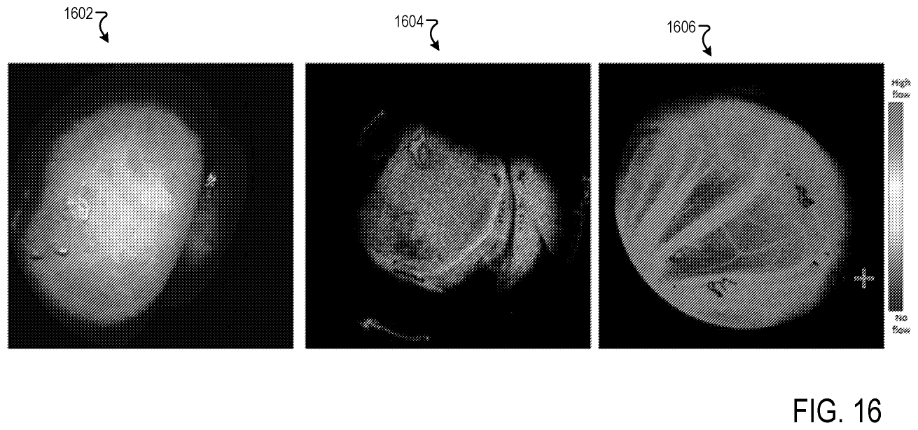

The pig study, described previously herein, shows that the system described herein, performs in a minimally invasive setting. LSCI imaging of various organs was achievable through a standard laparoscope with perfusion data displayed in real-time, as shown in FIG. 16. An area of the bowel is monitored. Clear show branching of the large vessels as well as smaller vasculature along the edges (image 1602 in FIG. 16). The back of the gallbladder is inspected and a vessel and its branch are identified (image 1604 in FIG. 16).

The pig mesentery is shown in image 1606 in FIG. 16. A notable point in image 1606 is the visualization of both small and large vessels. Typically, small blood vessels have a slower flow than large blood vessels, which is shown in image 1606—the small vessels have a lower signal than the large vessels. Because of difficulty judging distance in vivo, it is possible that the laparoscope was held further than 5 cm from the tissue, allowing light to expand more. This may indicate that the system described herein can operate further than the 5 cm observed in vitro given larger vessel sizes and environmental conditions. Furthermore, an increase of flow during cardiac pulsation is observed, which shows that despite illumination bias described previously herein, relative flow rates at the same position over time can be monitored.

The studies described herein show that the laparoscopic laser-speckle system described herein is capable of imaging the relative flow rates over time using the microfluidic phantom 700. The in vivo experiments illustrate the ability of the system described herein to highlight blood vessels (i.e., vessel identification) and determine changes in the perfusion levels before physical tissue changes develop, as well as its ability to be used in an MIS setting. Using a crossed-polarizer pair is effective for reducing the specular reflection from the tissue, yielding smoother results.

In the rat study described herein, the reflections cause shadows to appear on unpolarized acquisition, whereas polarized images are free of aberrations from reflection. Polarization may reduce the speckle contrast, decreasing the quality of the resultant images. Additionally, scattering from polarizer material and surface imperfections may further degrade the speckle quality.

In large-animal MIS, the application of LSCI becomes more difficult owing to the artifacts from increased organ movement relative to the laparoscope caused by breathing and pulsation. Additionally, the design of the laparoscope as a hand-held tool makes it more vulnerable to user movement and vibrations. In one implementation, the laparoscope 102 may be held in a fixed holder. Image registration has been proven to be capable of compensating for considerable motion, and in future studies this technique should be applied to the processing algorithm.

The laparoscope tip is not only small, but also contains a central lens stack to the camera surrounded by light fibers. Any lens fitted over the front of the laparoscope must have special geometry to not interfere with the optical path of the camera. For example, an aspherical lens array attached to the tip of a laparoscope capable of producing a more uniformly illuminated and expanded field may be used.

In one implementation, methods for computational compensation of illumination-related artifacts which may be used to minimize/avoid the drawbacks and design challenges of additional lens.

A system which includes the features in the foregoing description provides numerous advantages to users. The system and methodologies described herein holds promising advantages over other clinically available technologies for organ perfusion. Fluorescence angiography, for example, typically only provides a binary indication of the presence of perfusion in a region. The system described herein can provide visualization of the relative flow speeds which can be compared temporally. Time-to-peak of fluorescence signals during angiography can be related to perfusion levels, but requires a long imaging window for appropriate calculations. The system described herein is label-free and not temporally limited. The LSCI-processed images can be turned on and off at any time for any duration. This implies that the system offers increased flexibility and the ability to rapidly and continuously image areas over time to detect changes.

In one implementation, multi-exposure LSCI can be used. Multi-exposure improves the linearity of the system and has been demonstrated in real-time, providing measurements of the relative flow in environments with static scattering.

Laparoscopic LSCI has a strong potential for clinical application in intestinal surgery. The rigid laparoscopic real-time LSCI device with an integrated light source and working range of 5 cm provides the capability to continuously visualize vasculature and perfusion laparoscopically in real-time. The system may be used to enhance knowledge in the operating room and to improve intraoperative assessment of bowel tissue, leading to improved surgical outcomes. Because LSCI allows a non-invasive, label-free examination of vasculature and tissue perfusion, a distant common-path laparoscopic LSCI has a huge potential for various surgical applications. In addition, the system described herein may be used in semi-autonomous or fully autonomous robotic surgery.

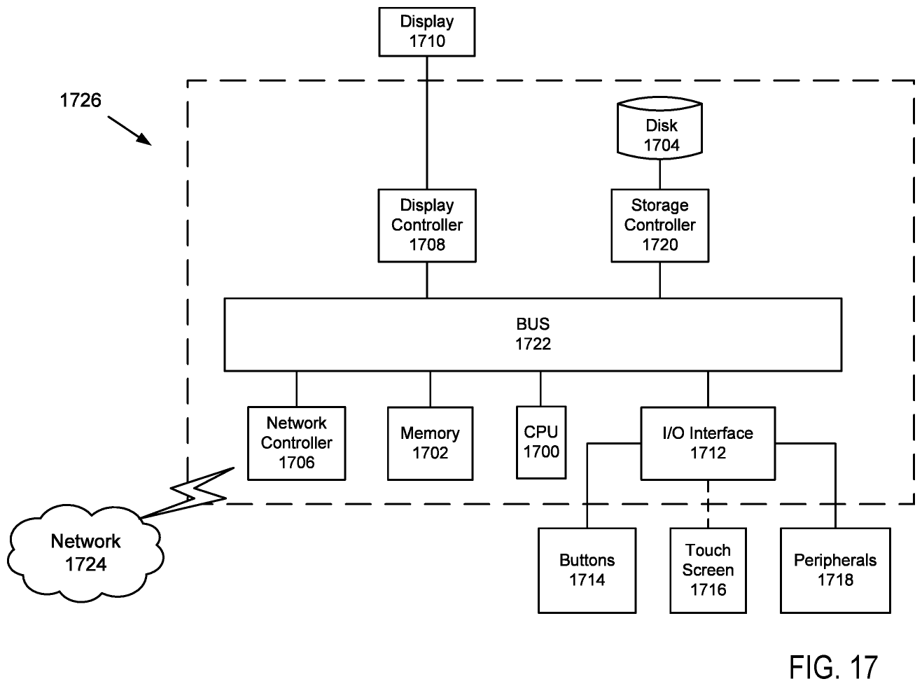

In one implementation, the functions and processes of the system 300 may be implemented by a computer 1726. Next, a hardware description of the computer 1726 according to exemplary embodiments is described with reference to FIG. 17. In FIG. 17, the computer 1726 includes a CPU 1700 which performs the processes described herein. The process data and instructions may be stored in memory 1702. These processes and instructions may also be stored on a storage medium disk 1704 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computer 1726 communicates, such as a server or computer.

Further, the claimed advancements may be provided as a utility application, background daemon, or component of an operating system, or combination thereof, executing in conjunction with CPU 1700 and an operating system such as Microsoft® Windows®, UNIX®, Oracle® Solaris, LINUX®, Apple macOS® and other systems known to those skilled in the art.

In order to achieve the computer 1726, the hardware elements may be realized by various circuitry elements, known to those skilled in the art. For example, CPU 1700 may be a Xenon® or Core® processor from Intel Corporation of America or an Opteron® processor from AMD of America, or may be other processor (processing circuitry) types that would be recognized by one of ordinary skill in the art. Alternatively, the CPU 1700 may be implemented on an FPGA, ASIC, PLD or using discrete logic circuits, as one of ordinary skill in the art would recognize. Further, CPU 1700 may be implemented as multiple processors cooperatively working in parallel to perform the instructions of the inventive processes described above.

The computer 1726 in FIG. 17 also includes a network controller 1706, such as an Intel Ethernet PRO network interface card from Intel Corporation of America, for interfacing with network 1724. As can be appreciated, the network 1724 can be a public network, such as the Internet, or a private network such as LAN or WAN network, or any combination thereof and can also include PSTN or ISDN sub-networks. The network 1724 can also be wired, such as an Ethernet network, or can be wireless such as a cellular network including EDGE, 3G and 4G wireless cellular systems. The wireless network can also be WiFi®, Bluetooth®, or any other wireless form of communication that is known.

The computer 1726 further includes a display controller 1708, such as a NVIDIA® GeForce® GTX or Quadro® graphics adaptor from NVIDIA Corporation of America for interfacing with display 1710, such as a Hewlett Packard® HPL2445w LCD monitor. A general purpose I/O interface 1712 interfaces with a keyboard and/or mouse 1714 as well as an optional touch screen panel 1716 on or separate from display 1710. General purpose I/O interface also connects to a variety of peripherals 1718 including printers and scanners, such as an OfficeJet® or DeskJet® from Hewlett Packard®.

The general purpose storage controller 1720 connects the storage medium disk 1704 with communication bus 1722, which may be an ISA, EISA, VESA, PCI, or similar, for interconnecting all of the components of the computer 1726. A description of the general features and functionality of the display 1710, keyboard and/or mouse 1714, as well as the display controller 1708, storage controller 1720, network controller 1706, and general purpose I/O interface 1712 is omitted herein for brevity as these features are known.

Obviously, numerous modifications and variations are possible in light of the above teachings. It is therefore to be understood that within the scope of the appended claims, the invention may be practiced otherwise than as specifically described herein.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

The above disclosure also encompasses the embodiments listed below.

(1) A visualization system including a laparoscope; a camera operatively coupled to the laparoscope; a light source operatively coupled to an illumination port of the laparoscope, the light source being configured to output one or more light beams each at a predetermined frequency to illuminate a target area; and processing circuitry configured to process imaging data and received by the camera to generate one or more images of the target area including at least one laser speckle contrast image, wherein the laparoscope is configured to output the one or more light beams toward the target area at a distal end thereof and to collect reflected and/or scattered light from the target area via the distal end.

(2) The visualization system according to (1), in which the laparoscope has an angle of from about zero to thirty degrees.

(3) The visualization system according to (1) or (2), in which the illumination port is a single illumination port and the laparoscope forms a common path for the imaging data received by the camera and the one or more light beams.

(4) The visualization system according to any of (1) to (3), in which the laparoscope is configured to be spaced from the target area from about 5 cm to about 10 cm when the target area is illuminated by the one or more light beams and the imaging data is received at the camera via the laparoscope.

(5) The visualization system according to any of (1) to (4), wherein at least one light beam of the one or more light beams is in an invisible range from near-infrared (NIR) to short-wave infrared laser (SWIR) and at least one light beam of the one or more light beams has a different frequency with respect to at least one other of the one or more light beams.

(6) The visualization system according to any of (1) to (5), in which the target area includes a tissue structure and the at least one light beam has a short wave infrared wavelength and the processing circuitry is configured to generate deep tissue information.

(7) The visualization system according to any of (1) to (6), in which the processing circuitry is configured to generate depth-resolved blood flow measurement.

(8) The visualization system according to any of (1) to (7), in which the light source includes a broadband visible light source and a near-infrared light source.

(9) The visualization system according to any of (1) to (8), in which the laparoscope is operatively coupled to the light source via a fiber optic light guide.

(10) The visualization system according to any of (1) to (9), in which the visualization system is configured for single-open, minimally invasive laparoscopic surgery and/or semi- or fully-autonomous surgery.

(11) The visualization system according to any of (1) to (10), in which at least one of the one or more light beams provided by the light source has a polarized pattern.

(12) The visualization system according to any of (1) to (11), in which the processing circuitry is further configured to perform real-time processing of the imaging data received by the camera.

(13) The visualization system according to any of (1) to (12), in which the processing circuitry further configured to generate a surgical scene for vasculature, tissue perfusion, and/other structures, including lymph nodes and tumor tissue.

(14) The visualization system according to any of (1) to (13), in which a view of the camera is focusable, has an adjustable field of view, is magnifiable, and/or has adjustable spatial resolution based on an adjustment of one of more of the laparoscope and the camera.

(15) The visualization system according to any of (1) to (14), further including a crossed-polarizer positioned between the distal end of the laparoscope and a sensor of the camera.

(16) The visualization system according to any of (1) to (15), further including an adjustable polarizer cap positioned at the distal end of the laparoscope.

(17) The visualization system according to any of (1) to (16), in which the laparoscope is set as a single laparoscope of the visualization system to illuminate lights and receive the imaging data by the camera such that no shadow areas are produced on the target area.

(18) The visualization system according to any of (1) to (17), in which the camera includes an RGB camera and a near-infrared camera, and the visualization system further comprising a beam splitter configured to divide an optical path to the RGB camera and the near-infrared camera.

(19) An apparatus for laser speckle contrast imaging, the apparatus including a laparoscope having an illumination port; and one or more image sensors operatively coupled to the laparoscope; in which the laparoscope is configured to receive one or more light beams via the illumination port, output the one or more light beams toward a target area, and capture one or more images of the target area via a common path.

(20) A visualization method including providing a visualization apparatus including a laparoscope, a camera operatively coupled to the laparoscope, a light source operatively coupled to an illumination port of the laparoscope; outputting one or more light beams generated by the light source at predetermined frequencies via the laparoscope to illuminate a target area; capturing reflected and/or scattered light from the target area via the laparoscope; and processing the captured light to generate at least a laser speckle contrast image of the target area.

1. A visualization system comprising:
a laparoscope;
a camera operatively coupled to the laparoscope;
a light source operatively coupled to an illumination port of the laparoscope, the light source being configured to output one or more light beams each at a predetermined frequency to illuminate a target area; and
processing circuitry configured to process imaging data received by the camera to generate one or more images of the target area including at least one laser speckle contrast image,
wherein the laparoscope is configured to output the one or more light beams toward the target area at a distal end thereof and to collect reflected and/or scattered light from the target area via the distal end.

2. The visualization system of claim 1, wherein the laparoscope has an angle of from about zero to thirty degrees.

3. The visualization system of claim 1, wherein the illumination port is a single illumination port and the laparoscope forms a common path for the imaging data received by the camera and the one or more light beams.

4. The visualization system of claim 1, wherein the laparoscope is configured to be spaced from the target area from about 5 cm to about 10 cm when the target area is illuminated by the one or more light beams and the imaging data is received at the camera via the laparoscope.

5. The visualization system of claim 1, wherein at least one light beam of the one or more light beams is in an invisible range from near-infrared (NIR) to short-wave infrared laser (SWIR) and at least one light beam of the one or more light beams has a different frequency with respect to at least one other of the one or more light beams.

6. The visualization system of claim 5, wherein the target area includes a tissue structure and the at least one light beam has a short wave infrared wavelength and the processing circuitry is configured to generate deep tissue information.

7. The visualization system of claim 5, wherein the processing circuitry is configured to generate depth-resolved blood flow measurement.

8. The visualization system of claim 1, wherein the light source includes a broadband visible light source and a near-infrared light source.

9. The visualization system of claim 1, wherein the laparoscope is operatively coupled to the light source via a fiber optic light guide.

10. The visualization system of claim 1, wherein the visualization system is configured for single-open, minimally invasive laparoscopic surgery and/or semi- or fully-autonomous surgery.

11. The visualization system of claim 1, wherein at least one of the one or more light beams provided by the light source has a polarized pattern.

12. The visualization system of claim 1, wherein the processing circuitry is further configured to perform real-time processing of the imaging data received by the camera.

13. The visualization system of claim 1, wherein the processing circuitry is further configured to generate a surgical scene for vasculature, tissue perfusion, and/other structures, including lymph nodes and tumor tissue.

14. The visualization system of claim 1, wherein a view of the camera is focusable, has an adjustable field of view, is magnifiable, and/or has adjustable spatial resolution based on an adjustment of one of more of the laparoscope and the camera.

15. The visualization system of claim 1, further comprising a crossed-polarizer positioned between the distal end of the laparoscope and a sensor of the camera.

16. The visualization system of claim 1, further comprising:
an adjustable polarizer cap positioned at the distal end of the laparoscope.

17. The visualization system of claim 1, wherein the laparoscope is set as a single laparoscope of the visualization system to illuminate lights and receive the imaging data by the camera such that no shadow areas are produced on the target area.

18. The visualization system of claim 1, wherein the camera includes an RGB camera and a near-infrared camera, and the visualization system further comprising a beam splitter configured to divide an optical path to the RGB camera and the near-infrared camera.

19. An apparatus for laser speckle contrast imaging, the apparatus comprising:
a laparoscope having an illumination port; and
one or more image sensors operatively coupled to the laparoscope;
wherein the laparoscope is configured to receive one or more light beams via the illumination port, output the one or more light beams toward a target area, and capture one or more images of the target area via a common path.

20. A visualization method comprising:
providing a visualization apparatus including a laparoscope, a camera operatively coupled to the laparoscope, a light source operatively coupled to an illumination port of the laparoscope;
outputting one or more light beams generated by the light source at predetermined frequencies via the laparoscope to illuminate a target area;
capturing reflected and/or scattered light from the target area via the laparoscope; and
processing the captured light to generate at least a laser speckle contrast image of the target area.
